(19) United States
(12) Patent Application Publication
(10) Pub. No.: US 2017/0146554 A1
(43) Pub. Date: May 25, 2017

(54) METHOD FOR TEST ON DIABETIC NEPHROPATHY

(71) Applicants: Masanori HARA, Niigata (JP); Niigata University, Niigata (JP); Juntendo Educational Foundation, Tokyo (JP); Denka Seiken Co. Ltd., Tokyo (JP)

(72) Inventors: Masanori HARA, Niigata (JP); Akihiko SAITO, Niigata (JP); Yasuhiko TOMINO, Tokyo (JP); Katsuhiko ASANUMA, Tokyo (JP); Hiroyuki KUROSAWA, Niigata (JP); Shinya OGASAWARA, Niigata (JP); Yoshiaki HIRAYAMA, Niigata (JP)

(73) Assignees: Masanori HARA, Niigata (JP); Niigata University, Niigata (JP); Juntendo Educational Foundation, Tokyo (JP); Denka Seiken Co. Ltd., Tokyo (JP)

(21) Appl. No.: 15/401,698

(22) Filed: Jan. 9, 2017

Related U.S. Application Data
(63) Continuation-in-part of application No. 13/377,509, filed on Mar. 9, 2012, now abandoned.

(30) Foreign Application Priority Data
Jun. 10, 2009 (JP) 2009-139188

(51) Int. Cl. G01N 33/68; A61K 31/549; A61K 38/28

(52) U.S. Cl. CPC G01N 33/6893; A61K 38/28; A61K 31/549; G01N 2333/70596; G01N 2800/347; G01N 2800/56

(57) ABSTRACT

Provided is a test method for the detection of diabetic nephropathy at an early stage as compared to a conventional method. Specifically provided are: a test method for diabetic nephropathy, including detecting urinary podocalyxin; the test method, further including assessing diabetic nephropathy at at least Stage I; a test reagent for use in the test method; and a test reagent kit for use in the test method. The present invention is based on a finding that urinary podocalyxin reflects the development and condition of diabetic nephropathy with high sensitivity at an early stage as compared to urinary albumin.

### CROSS-REFERENCE TO RELATED APPLICATIONS

The present application is a Continuation-in-part of Ser. No. 13/377,509, which is a National Phase 371 application of PCT/JP2010/003837, filed Jun. 9, 2010, which claims priority from Japanese Patent Application No. 2009-139188, filed Jun. 10, 2009, which is incorporated herein by reference.

### TECHNICAL FIELD

The present invention relates to a test method for diabetic nephropathy, including detecting urinary podocalyxin, and a test reagent for diabetic nephropathy for use in the test method, including an anti-podocalyxin antibody.

The present application claims priority from Japanese Patent Application No. 2009-139188, which is incorporated herein by reference.

### BACKGROUND ART

Diabetic nephropathy is one of the three major complications of diabetes, and has been the leading cause for initiation of hemodialysis in recent years. Diabetic nephropathy develops when, ten or more years after the onset of diabetes, the diabetes is poorly controlled and a hyperglycemic condition lasts for a long period of time. An increase in glomerular filtration rate, an increase in intraglomerular pressure (glomerular hypertension), and an increase in permeability of a plasma protein from the glomerular capillaries lead to the occurrence of albuminuria. The further progression of the condition loosens the network of the glomerular basement membrane, and thus even a protein having a larger size starts to be excreted, resulting in proteinuria. Severe proteinuria leads to hypoproteinemia, which causes edema synergistically with a decrease in glomerular filtration function. The still further progression of the condition impairs the excretion of body wastes, water, and salts, resulting in a renal failure condition, which finally requires hemodialysis or renal transplantation.

The proportion and number of patients with diabetic nephropathy as the primary disease of end-stage renal failure have been increasing year by year, and the diagnosis and treatment of diabetic nephropathy are large problems. A dialysis patient with diabetic nephropathy often has a poor prognosis, and a dialysis therapy does not necessarily make a large contribution to the life prognosis and QOL improvement of the patient at present. Further, the clinical diagnosis of diabetic nephropathy is performed based on findings of persistent proteinuria, renal dysfunction, hypertension, and the like complicated with diabetes because of a difficulty of renal biopsy. However, once persistent proteinuria occurs, it is difficult to treat the condition, resulting in end-stage renal failure in many cases after a lapse of five to six years. Therefore, from the standpoint of clinical medicine, there is a strong demand for the diagnosis and treatment of renal lesions at an early stage before a urine protein is judged as positive in a conventional dipstick test.

As a technique for diagnosing a patient with diabetic nephropathy at an early stage at which a urine protein is judged as negative in the dipstick test, there is performed urinary albumin measurement using radioimmunoassay, enzyme-linked immunoassay, latex agglutination immunoturbidimetry, or immunoprecipitation. As an indicator for such technique, a urinary albumin excretion rate “ALB/Cre” is used. The urinary albumin excretion rate (mg/g of Cre) can be determined with 100×urinary albumin concentration (mg/L)/urinary creatinine concentration (mg/dL). A case where the urinary albumin excretion rate “ALB/Cre” is less than 30 mg/g of Cre corresponds to Stage I (pre-nephropathy), a case where the urinary albumin excretion rate is 30 to less than 300 mg/g of Cre corresponds to Stage II (incipient nephropathy), and a case where the urinary albumin excretion rate is 300 mg/g of Cre or more corresponds to Stage III (overt nephropathy). A case where the urinary albumin excretion rate is less than 30 mg/g of Cre is assessed to correspond to a stage at which nephropathy has not developed. When a subject to be tested (hereinafter, sometimes referred to as “subject”) shows microalbumin, the subject is diagnosed to have diabetic nephropathy, which corresponds to Stage II, a stage at which pathologically mild to moderate diffuse lesions have already existed and the presence of nodular lesions is also known. Thus, real initial nephropathy cannot necessarily be judged by the diagnosis of urinary albumin, and there is a demand for an indicator capable of diagnosing diabetic nephropathy at an early stage.

There is disclosed a method involving using, as a diagnostic indicator, a von Willebrand factor cleaving enzyme in place of the urinary albumin concentration (Patent Literature 1). Specifically, regarding the amount or enzymatic activity of the von Willebrand factor cleaving enzyme, there is disclosed a measure for predicting the degree of progression of renal injury and improving a prognosis in a patient with diabetic nephropathy by analyzing the amount of vWF using an immunological technique.

There is disclosed a simple test measure for renal injury involving measuring urinary podocalyxin in association with renal diseases (Patent Literature 2). Podocalyxin is a sugar protein which is present in the surface of podocytes constructing the renal glomerulus and is responsible for a filtration function. The podocytes are located on the Bowman's space side in the glomerular basement membrane and play important roles in the mechanism of glomerular filtration. Thus, it is known that the grasping of the degree of injury in the podocytes has an extremely important meaning in understanding renal diseases (Non Patent Literature 1).

### CITATION LIST

### Patent Literature

[PTL 1] JP 2008-216137 A
[PTL 2] WO 2002/037099 A1

### Non Patent Literature

[NPL 1] Hara et al., Nephron 69: 397-403 (1995)

### SUMMARY OF INVENTION

### Technical Problem

An object of the present invention is to provide a test method for the detection of diabetic nephropathy at an early stage as compared to a conventional method.

### Solution to Problem

In order to solve the above-mentioned problem, the inventors of the present invention have focused on the fact that, when a subject has a urinary podocalyxin excretion rate “PCX/Cre” higher than a value therefor in a healthy subject, the subject is suspected of having diabetic nephropathy, and have found that diabetic nephropathy can be tested by detecting urinary podocalyxin. Thus, the present invention has been completed.

That is, the present invention includes the following items.

1. A test method for diabetic nephropathy, including detecting urinary podocalyxin.

2. A test method according to the item 1, in which a stage of the diabetic nephropathy is classified into at least Stage I.

3. A test method according to the item 1 or 2, further including assessing a subject to be tested who has a urinary podocalyxin value higher than a reference value to have diabetic nephropathy.

4. A test method according to the item 3, in which the reference value includes an upper limit value of a 95% confidence interval of a urinary podocalyxin value in a healthy subject.

5. A test method for diabetic nephropathy according to the items 1 or 2, further including assessing progression of the diabetic nephropathy and/or staging of the diabetic nephropathy.

6. A test method according to any one of the items 1 to 5, in which the urinary podocalyxin value is corrected with a value for a urinary component.

7. A test method according to the item 6, in which the urinary component includes urinary creatinine.

8. A test method according to any one of the items 1 to 7, in which the detecting of the urinary podocalyxin is carried out by an immunological technique.

9. A test reagent for diabetic nephropathy for use in the test method of the item 8, including an anti-podocalyxin antibody for detecting urinary podocalyxin.

10. A test reagent kit for diabetic nephropathy for use in the test method of the item 8, including a reagent for detecting urinary podocalyxin using an anti-podocalyxin antibody.

### Advantageous Effects of Invention

The test method of the present invention can be performed by measuring urinary podocalyxin, thereby detecting diabetic nephropathy at an early stage. The test method of the present invention allows even a patient classified into Stage I (pre-nephropathy) with a urinary albumin excretion rate “ALB/Cre” to be reclassified into a patient at a high risk of nephropathy. The detection of diabetic nephropathy at an early stage by the test method of the present invention contributes to the prevention of nephropathy progression as well.

Further, the urinary podocalyxin excretion rate “PCX/Cre” tends to increase along with the progression of diabetic nephropathy, and the condition of diabetic nephropathy can be grasped by the test method of the present invention. The urinary podocalyxin excretion rate reflects glomerular hyperfunction and the subsequent progressive injury, and hence can be used for effectively checking active glomerular injury. It is conceivable that the test method of the present invention can simply monitor the condition of diabetic nephropathy and rapidly determine a therapeutic strategy or the like.

### BRIEF DESCRIPTION OF DRAWINGS

FIG. 1 is a graph illustrating urinary podocalyxin excretion rates “PCX/Cre” of patients with diabetes and diabetic nephropathy classified in accordance with the staging of diabetic nephropathy (Example 2).

FIG. 2 is a graph illustrating urinary podocalyxin excretion rates “PCX/Cre” of patients with diabetes and diabetic nephropathy except those with eGFR<60 classified in accordance with the staging of diabetic nephropathy (Example 2).

### DESCRIPTION OF EMBODIMENTS

As used herein, the term “treating”, unless otherwise stated, refers to reversing, relieving or inhibiting the progression or preventing diseases or disorders to which the above term is applied or at least one symptom thereof. As used herein, the term “treatment” refers to an act of treating when “treating” is defined as above.

The present invention includes testing diabetic nephropathy by detecting urinary podocalyxin of a subject. In this description, urine, which is used as a specimen, may be obtained from any subject, but is preferably obtained from a patient suffering from diabetes. The diagnosis of diabetes has only to be performed by a conventionally known measure. No particular limitation is imposed on a collection method for urine, but it is preferred to use early morning urine or casual urine. Further, the amount of urine necessary for the test method of the present invention is about 10 to 200 μL. The test method of the present invention may be performed concurrently with a general urine test to be conventionally performed in a medical examination or the like, or may be performed by separately collecting urine from a subject suspected of having diabetes or a patient judged to have diabetes. Further, the test method of the present invention may be performed for the purpose of monitoring the development or progression of diabetic nephropathy in a patient with diabetes.

Urine as a specimen may be treated by adding and mixing a treatment liquid into the urine. The treatment liquid may be any as long as the pH adjustment of the urine, the masking of a urine sediment, and the solubilization of podocalyxin are possible, but is preferably exemplified by a solution obtained by adding a chelating agent, a surfactant, and the like to a buffer. The buffer and the chelating agent may be any known buffer and chelating agent, and it is preferred to use a nonionic surfactant as the surfactant. The treatment liquid is exemplified by a solution including 0.2 M EDTA and 2% (Vol./Vol.)

Triton X-100 in 2 M TES-NaOH (pH 7.0). A urine sample solution can be obtained by adding and mixing 10 μL of such treatment liquid into 90 μL of a urine specimen.

Various methods may be employed as a detection method for podocalyxin in the urine sample solution. An example of the detection method for the urinary podocalyxin is an immunological technique. The immunological technique may be performed, for example, by an immunostaining method (including a fluorescent antibody method, an enzymatic antibody method, a heavy metal-labeled antibody method, and a radioisotope-labeled antibody method), a combination of separation based on an electrophoresis method and a detection method with fluorescence, an enzyme, a radioisotope, or the like (including a western blot method and a fluorescent two-dimensional electrophoresis method), enzyme-linked immunosorbent assay (ELISA), a dot blotting method, latex agglutination-turbidimetric immunoassay (LA), or immunochromatography. Of those, it is preferred to employ an ELISA method or an LA method. It is preferred to employ a sandwich method in the ELISA method from the viewpoint of quantitative property. In the sandwich method, a urine sample solution is added to an anti-podocalyxin antibody-coated microtiter plate to cause an antigen-antibody reaction, an enzyme-labeled anti-podocalyxin antibody is further added to cause an antigen-antibody reaction, the plate is washed and then subjected to a reaction with an enzyme substrate and color development, the absorbance is measured to detect urinary podocalyxin, and the measured value can be used to calculate a urinary podocalyxin concentration.

The anti-podocalyxin antibody for use in the immunological technique has only to be an antibody capable of detecting podocalyxin. The anti-podocalyxin antibody for use in the present invention is not particularly limited, and may be a known antibody or an antibody to be developed in the future. Examples thereof include monoclonal and polyclonal antibodies, a labeled antibody, a chimeric antibody, a humanized antibody, and binding active fragments thereof.

A value for urinary podocalyxin for use in the present invention may be a urinary podocalyxin concentration, but is desirably a urinary podocalyxin concentration corrected with a value for a urinary component to be stably excreted in urine (urinary component value). The urinary component is particularly preferably urinary creatinine. It is considered that urinary creatinine is substantially constant irrespective of a disease in one individual because the production of creatinine depends on the amount of a muscle. In a test for a urinary excretion substance, in order to eliminate an error in urinary amount, a technique involving correcting the amount of a urinary excretion substance of interest with an amount per g of creatinine is generally employed. This allows the comparison of urinary excretion substances per unit gram of creatinine. A corrected value obtained by correcting a urinary podocalyxin concentration with a urinary creatinine concentration is referred to as urinary podocalyxin excretion rate (PCX/Cre), and the urinary podocalyxin excretion rate can be calculated with the following equation.

PCX/Cre: urinary podocalyxin excretion rate (μg/g)=100×urinary podocalyxin concentration (ng/mL)/urinary creatinine concentration (mg/dL) <Equation>

When a subject has a urinary podocalyxin value, preferably a urinary podocalyxin excretion rate “PCX/Cre”, which is obtained by the test method of the present invention, higher than a reference value, the subject can be assessed to have diabetic nephropathy. The reference value may be appropriately set, but a urinary podocalyxin value, preferably a urinary podocalyxin excretion rate “PCX/Cre” in a healthy subject may be used. The healthy subject may include a patient with diabetes who has not developed diabetic nephropathy as well as a subject except a patient with diabetes. However, it is preferred that the healthy subject be desirably a subject except a patient with diabetes, more desirably a subject negative for an additional renal function marker except a patient with diabetes. Examples of the additional renal function marker include an estimated glomerular filtration rate (eGFR) and a urine protein. It is more preferred that urinary podocalyxin values, preferably urinary podocalyxin excretion rates “PCX/Cre” be determined from a plurality of healthy subjects and the upper limit value of the 95% confidence interval of the urinary podocalyxin excretion rates be used as a reference value.

The 95% confidence interval may be determined by a known technique. When urinary podocalyxin values in healthy subjects follow a normal distribution, the 95% confidence interval thereof can be determined with the following equation.

95% confidence interval=average of urinary podocalyxin values in healthy subjects±t×standard deviation of urinary podocalyxin values in healthy subjects

It should be noted that t represents a degree of freedom and varies depending on the number of specimens of healthy subjects, and hence has only to be selected based on a t-distribution table. In general, t represents 1.96 in the case of the 95% confidence interval.

Meanwhile, when urinary podocalyxin values in healthy subjects do not follow a normal distribution, a range that accounts for 95% including the median is defined as a reference range, and the upper limit value of the reference range is defined as a reference value for the urinary podocalyxin values.

In the present invention, the reference value is 50 μg/g to 300 μg/g, preferably 75 μg/g to 200 μg/g, more preferably 100 μg/g to 180 μg/g.

The stages of diabetic nephropathy are generally classified into Stage I (pre-nephropathy), Stage II (incipient nephropathy), and Stage III (overt nephropathy). In Stage I (pre-nephropathy), a urinary albumin value is normal, and as histopathological features, no or mild diffuse lesions exist. In Stage II (incipient nephropathy), it is known that microalbuminuria occurs, and as histopathological features, mild to moderate diffuse lesions exist and nodular lesions exist in some cases. In Stage III (overt nephropathy), it is known that persistent proteinuria occurs, and as histopathological features, moderate to severe diffuse lesions exist and nodular lesions exist in many cases.

The test method of the present invention can assess diabetic nephropathy at Stage I as well as Stage II and Stage III in the above-mentioned staging. In the case of conventional staging with urinary albumin measurement, Stage I is assessed as a stage at which nephropathy has not developed. In contrast, the test method of the present invention can assess Stage I, which allows diabetic nephropathy to be diagnosed at an early stage.

Further, the urinary podocalyxin excretion rate “PCX/Cre” reflects glomerular hyperfunction under progression and the subsequent progressive injury. Therefore, an increase in the urinary podocalyxin excretion rate can be confirmed to assess and/or predict the progression of the condition of diabetic nephropathy. When the progression of the condition of diabetic nephropathy is assessed and/or predicted, it is preferred to measure a urinary podocalyxin value in a subject with time, and an increase in the urinary podocalyxin value is assessed as the progression of the condition of diabetic nephropathy. Further, the staging of diabetic nephropathy may also be performed based on the urinary podocalyxin excretion rate.

A method involving assessing and/or predicting the progression of the condition of diabetic nephropathy or performing the staging of diabetic nephropathy with use of glomerular hyperfunction under progression is preferably applied to a subject who has active glomeruli and is free of advanced glomerulosclerosis. Such subject is preferably a subject negative for an additional renal function marker, and is exemplified by a subject with eGFR of 60 or more.

The present invention also encompasses a test reagent for diabetic nephropathy for use in the test method, including an anti-podocalyxin antibody for detecting urinary podocalyxin, and a test reagent kit for diabetic nephropathy for use in the test method, including a reagent including an anti-podocalyxin antibody. The anti-podocalyxin antibody included in the test reagent or the test reagent kit may be labeled, for example, with an enzyme or the like. Further, the test reagent kit may include two or more kinds of anti-podocalyxin antibodies, and the antibodies are preferably antibodies capable of recognizing epitopes different from each other. In addition, the kit may include a reagent such as a treatment liquid or a chromogenic substrate, an instrument necessary for a test, and the like.

In a further aspect, the invention relates to a method of treating or selecting a treatment of diabetic nephropathy, preferably Stage 1 diabetic nephropathy in a subject suffering from the condition.

The treatment method may include administering medication to the subject suffering diabetic nephropathy. Such medicine may include without limitation, hypoglycemic agent, antihypertensive agent, and/or diuretic. The hypoglycemic agent may include without limitation, insulin. The antihypertensive agent may include without limitation, angiotensin-converting enzyme inhibitor, an angiotensin receptor antagonist, and/or calcium blocker. The angiotensin-converting enzyme inhibitor may include without limitation, Perindopril, Delapril, Trandolapril, Cilazapril, Captopril, Enalapril, Lisinopril, Benazepril, or Imidapril. The angiotensin receptor antagonist may include without limitation, Losartan, Valsartan, Candesartan, Telmisar, Olmesartanmedoxomil, Irbesartan, or Azilsartan. The calcium blocker may include without limitation, Nifedipine, Amlodipine, or Diltiazem hydrochloride. The diuretic may be without limitation, Trichlormethiazide.

In an alternative aspect, the treatment method may include other non-medicinal treatment or therapeutic protocol. These treatment or therapy may include, without limitation, blood sugar management, weight reduction, diet therapy, and/or simply resting.

In a method of selecting a subject for the treatment of diabetic nephropathy, the method may include determining the level of podocalyxin in the urine of candidate subject.

The level of podocalyxin found in the urine of the candidate subject can be used to assess the level of diabetic nephropathy. Such information is very valuable to a clinician, to determine a treatment protocol for diabetic nephropathy. Information on how severe the diabetic nephropathy condition is can be used to determine whether further treatment is required. Such a treatment may include any of the treatment or therapy listed above.

The invention also relates to methods for assessing the severity of diabetic nephropathy in a subject, which includes assessing the amount of podocalyxin found in the urine of the subject. The term “severity” with respect to diabetic nephropathy refers to the Stage 1, 2 and so forth, where the characteristics of each stage is well known in the art. The severity of diabetic nephropathy is often accompanied by the amount of podocalyxin released into urine.

The invention also relates to a method for monitoring the effectiveness of treatment of diabetic nephropathy, wherein the method comprises detecting the level of podocalyxin present in urine, and determining the effectiveness of a treatment. The level of presence of podocalyxin can be monitored over the course of a treatment period. As the level of podocalyxin present in urine provides an indication of the severity of diabetic nephropathy, monitoring the amount of podocalyxin over the course of a treatment can indicate the effectiveness of the treatment.

In all of the methods described above, including methods of treatment of diabetic nephropathy, a key step is detecting the amount of podocalyxin in urine. In one aspect, the method includes (i) adding a treatment liquid comprising a chelating agent to a urine sample of a subject to produce a urine sample solution comprising solubilized podocalyxin; (ii) detecting the level determining an amount or concentration of solubilized podocalyxin in the urine sample solution as a value for a urinary podocalyxin; (iii) calculating a urinary podocalyxin value as a diagnostic marker, wherein the amount or concentration of urinary solubilized podocalyxin is corrected with a value an amount or concentration for of urinary creatinine; and (iv) determining a therapeutic strategy of diabetic nephropathy for a subject who has the urinary podocalyxin value higher than a reference value, and treating the subject suffering from diabetic nephropathy according to a therapeutic strategy.

The pharmaceutical composition or medicinal formulation for treating diabetic nephropathy, preferably Stage 1, according to the present invention can be used in different types of oral formulations such as powders, granules, tablets, capsules, suspensions, emulsions, syrups, aerosols, and external application, suppository, and sterilized injection solution each of which by a commonly employed method.

For suitable carriers, excipients, and diluents that can be included in the composition including the extract of the present invention, there are lactose, dextrose, sucrose, sorbitol, mannitol, xylitol, erythritol, maltitol, starch, acacia rubber, alginate, gelatin, calcium, phosphate, calcium silicate, cellulose, methyl cellulose, crude cellulose, polyvinyl pyrrolidone, water, methylhydroxybezoate, propyl hydroxybenzoate, talc, magnesium stearate, and mineral oils.

If it is formulated, normally used diluents or excipients such as filler, extender, binder, humectant, disintegrator, and surfactant are used in preparation.

Solid formulations for oral administration include tablets, pills, powders, granules, and capsules and for these solid formulations are prepared by adding at least one excipient to compound above, for example, starch, and calcium carbonate, sucrose, or lactose, gelatin. Additionally, lubricants such as magnesium steareate, and talc are also used besides simple excipient. As liquid formulations for oral intake, there are suspension, solution, emulsion and syrup and a number of different excipients for example, humectants, sweeteners, aromatics, and preservatives can be included besides water, a simple diluents, and liquid paraffin.

As formulations for parenteral administration, sterilized aqueous solutions, non-aqueous solvents, suspensions, emulsions, lyophilized preparations, and suppositories are included. For non-aqueous solvents and suspensions, vegetable oil such as propylene glycol, polyethylene glycol, and olive oil and injectable ester such as ethyl oleate can be used. Witepsol, macrogol, Tween 61, cacao butter, laurinum, and glycerogelatin can be used as suppository bases.

The diabetic nephropathy treatment composition of the present invention varies depending on the age, gender and weight of subject, but 0.0001 to 100 mg/kg, preferably 0.001 to 10 mg/kg can be administered once to several times in a day. Moreover, the dosage can be increased or decreased according to the route of administration, severity of diseases, gender, weight and age. Therefore, the scope of the present invention is not limited at any aspect by the above-described dosage.

The above-described pharmaceutical composition can be administered through a variety of routes. All methods of administration are predictable, for example, oral, rectal, or intravenous, intramuscular, subcutaneous, intrauterine, or cerebrovascular injection.

The form of pharmacological administration of the pharmaceutical composition of the present invention can be used as pharmacologically acceptable form of salts and also it can be used independently or combined with other pharmacologically active compounds as well as appropriate combination thereof.

### EXAMPLES

Hereinafter, the present invention is further specifically described by way of examples of the present invention. However, the present invention is by no means limited thereto, and various applications are possible without departing from the technical idea of the present invention.

### (Example 1) Measurement of Urinary Podocalyxin Concentration

A podocalyxin concentration was measured using two kinds of anti-human podocalyxin monoclonal antibodies. Those two kinds of antibodies recognize different two epitopes of human podocalyxin, respectively, and are an anti-human podocalyxin monoclonal antibody a (hereinafter, simply referred to as “antibody a”) and an anti-human podocalyxin monoclonal antibody b (hereinafter, simply referred to as “antibody b”), respectively. In this example, an antibody a-coated microtiter plate (split type micro plate GF8 high: Nunc) and a horseradish peroxidase (hereinafter, abbreviated as “HRP”)-labeled antibody b were used.

First, 90 μL of urine obtained from a subject were mixed with 10 μL of a solution of 2 M TES-NaOH, 0.2 M EDTA, and 2% (Vol./Vol.) Triton X-100, pH 7.0. 100 μL of a urine sample solution obtained by the mixing were added to wells of an antibody a-coated microtiter plate. The plate was left to stand still at 37° C. for 1 hour, and the urine sample solution was then removed by decantation from the wells. Washing was performed by adding 3.6 mM Na₂HPO₄, 1.4 mM KH₂PO₄, 145 mM NaCl, and 0.05% (Vol./Vol.) Tween 20 (hereinafter, abbreviated as “PBS-T”) to the wells of the microtiter plate at 200 μL/well and removing PBS-T by decantation. The washing step was performed a total of three times. After that, an HRP-labeled antibody b solution was added at 100 μL/well. The plate was left to stand still at 37° C. for 1 hour, and the HRP-labeled antibody b solution was then removed by decantation. Washing was performed by adding PBS-T at 200 μL/well and removing PBS-T by decantation. The washing step was performed a total of three times. After that, a TMB One-Step Substrate System (Dako) was used as a substrate solution for an HRP enzymatic reaction and added at 100 μL/well, and the plate was left to stand still under a light-shielding condition at 25° C. for 30 minutes. After that, a 313 mM H₂SO₄ solution was added at 100 μL/well as a reaction terminating solution, and each of the wells was measured for its absorbances at wavelengths of 450 nm and 630 nm using Multiskan Ascent and Ascent Software for Multiskan (Dainippon Pharmaceutical Co., Ltd.). Then, a value obtained by subtracting the absorbance at a wavelength of 630 nm from the absorbance at a wavelength of 450 nm was defined as a measured value. Native human podocalyxin extracted from the kidney was used as a standard for a calibration curve to derive a podocalyxin concentration in a specimen. Then, a urinary podocalyxin excretion rate, in which a urinary podocalyxin concentration was corrected with a urinary creatinine concentration, was calculated with the following equation.

PCX/Cre: urinary podocalyxin excretion rate (μg/g)=100×urinary podocalyxin concentration (ng/mL)/urinary creatinine concentration (mg/dL) <Equation>

### (Example 2) Clinical Significance of Urinary Podocalyxin Excretion Rate in Diabetic Nephropathy

The urinary podocalyxin excretion rates (creatinine-corrected values) of 71 patients with diabetes and diabetic nephropathy were determined by the method of Example 1 and classified in accordance with the staging of diabetic nephropathy. A group of healthy subjects is defined as Group A, a group of patients with diabetic nephropathy at Stage I (pre-nephropathy) is defined as Group B, a group of patients with diabetic nephropathy at Stage II (incipient nephropathy) is defined as Group C, and a group of patients with diabetic nephropathy at Stage III (overt nephropathy) is defined as Group D. This means that a specimen, in which the urinary podocalyxin excretion rate “PCX/Cre” determined by the method of Example 1 showed a higher value than a reference value, has diabetic nephropathy. The reference value is the upper limit value of the 95% confidence interval of urinary podocalyxin excretion rates “PCX/Cre” determined from urine collected from 66 healthy subjects (excluding a patient with diabetes in this example). The reference value is 161 μg/g.

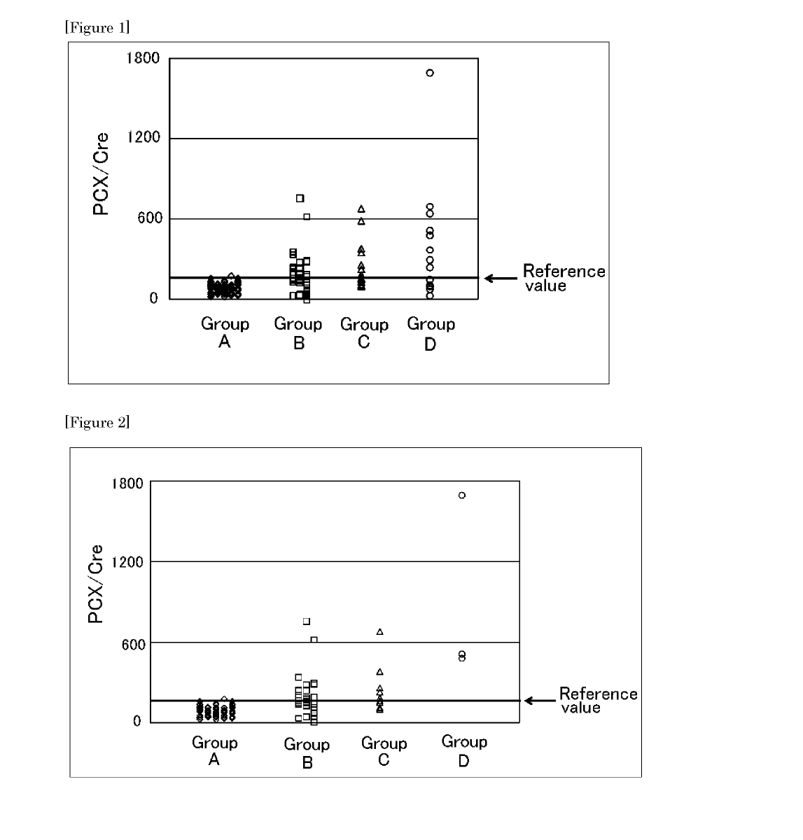

Table 1 and FIG. 1 show the urinary podocalyxin excretion rates “PCX/Cre” of patients with diabetes and diabetic nephropathy classified in accordance with the staging of diabetic nephropathy.

TABLE 1

| | | Diabetic nephropathy | | |
|---|---|---|---|---|
| | | | Stage II | Stage III |
| | Healthy | Stage I | (incipient | (overt |
| | subject | (pre-nephropathy) | nephropathy) | nephropathy) |
| Urinary podocalyxin | 1/66 | 18/39 | 8/17 | 9/15 |
| excretion rates equal to | cases | cases | cases | cases |
| or more than reference | | | | |
| value | | | | |
| (Number of cases) | | | | |
| Urinary podocalyxin | 1.5% | 46.2% | 47.1% | 60.0% |
| excretion rates equal to | | | | |
| or more than reference | | | | |
| value | | | | |
| (%) | | | | |

Table 1 and FIG. 1 revealed that the percentage of specimens having urinary podocalyxin excretion rates “PCX/Cre” equal to or more than the reference value became higher as the condition got worse. The table and figure also revealed that patients having urinary podocalyxin excretion rates “PCX/Cre” equal to or more than the reference value had been present at 46.2% at the normoalbuminuric stage (Stage I) before the occurrence of microalbuminuria. This means that podocalyxin has been excreted in urine before the occurrence of microalbuminuria. The results revealed that the detection of urinary podocalyxin allowed a patient classified into Stage I (pre-nephropathy) in accordance with the staging of diabetic nephropathy to be reclassified into a patient at a high risk of nephropathy.

Next, examination was made as to whether or not the urinary podocalyxin excretion rate “PCX/Cre” was able to be used for checking active glomerular injury. For that purpose, among the 71 patients with diabetes and diabetic nephropathy, 46 patients except patients with eGFR<60 estimated to have advanced glomerulosclerosis and to have a decreased gap space on the Bowman's space side capable of allowing urinary excretion were each subjected to sub-analysis for their urinary podocalyxin excretion rate “PCX/Cre.”

Table 2 and FIG. 2 show the urinary podocalyxin excretion rates “PCX/Cre” of the patients with diabetes and diabetic nephropathy except those with eGFR<60 classified in accordance with the staging of diabetic nephropathy.

TABLE 2

| | | Diabetic nephropathy | | |
|---|---|---|---|---|
| | | | Stage II | Stage III |
| | Healthy | Stage I | (incipient | (overt |
| | subject | (pre-nephropathy) | nephropathy) | nephropathy) |
| Urinary podocalyxin | 1/66 | 15/33 | 6/10 | 3/3 |
| excretion rates equal to | cases | cases | cases | cases |
| or more than reference | | | | |
| value | | | | |
| (number of cases) | | | | |
| Urinary podocalyxin | 1.5% | 45.5% | 60.0% | 100.0% |
| excretion rates equal to | | | | |
| or more than reference | | | | |
| value | | | | |
| (%) | | | | |

As seen from a comparison between the results of the 71 patients with diabetes and diabetic nephropathy in the staging of diabetic nephropathy and the results of the 46 patients except the patients with eGFR<60, the percentages of patients having urinary podocalyxin excretion rates “PCX/Cre” equal to or more than the reference value are equivalent in both the patients at Stage I, whereas much higher values are observed in the 46 patients except the patients with eGFR<60 at Stage II and Stage III (Table 1, Table 2, FIG. 1, and FIG. 2). In other words, the comparison revealed that the urinary podocalyxin excretion rate reflected glomerular hyperfunction under progression and the subsequent progressive injury. In other words, a test method suitable for checking active glomerular injury can be provided by the use of the urinary podocalyxin excretion rate “PCX/Cre.”

### INDUSTRIAL APPLICABILITY

As described above, the urinary podocalyxin excretion rate “PCX/Cre” was found to reflect the development of diabetic nephropathy with high sensitivity at an early stage as compared to the urinary albumin excretion rate “ALB/Cre,” which has been used as a diagnostic indicator for diabetic nephropathy. Further, the use of the urinary podocalyxin excretion rate “PCX/Cre” as a diagnostic marker for diabetic nephropathy can detect diabetic nephropathy at an early stage and accurately determine the degree of injury (progression of the condition). The test method of the present invention is useful because the method can prevent diabetic nephropathy from advancing in severity, reduce a medical treatment cost, and make a rapid decision on a therapeutic strategy or the like.

1. A method for treating diabetic nephropathy, comprising treating diabetic nephropathy in a subject, comprising testing for diabetic nephropathy in the subject, comprising the steps of:
(i) adding a treatment liquid comprising a chelating agent to a urine sample of a subject to produce a urine sample solution comprising solubilized podocalyxin;
(ii) determining an amount or concentration of solubilized podocalyxin in said urine sample solution;
(iii) calculating a urinary podocalyxin value as a diagnostic marker, wherein the amount or concentration of solubilized podocalyxin is corrected with an amount or concentration of urinary creatinine; and
(iv) determining a therapeutic strategy of diabetic nephropathy for a subject who has the urinary podocalyxin value higher than a reference value, and treating the subject suffering from diabetic nephropathy according to a therapeutic strategy.

2. The method according to claim 1, wherein the diabetic nephropathy is classified at Stage I.

3. The method of claim 1, wherein the reference value is an upper limit value of a 95% confidence interval of a urinary podocalyxin value in healthy subjects,
wherein the urinary podocalyxin value is determined from a plurality of healthy subjects and the upper limit value of the 95% confidence interval of the urinary podocalyxin value is used as a reference value,
wherein the 95% confidence interval is determined with the following equation:
95% confidence interval=average of urinary podocalyxin values in healthy subjects±t×standard deviation of urinary podocalyxin values in healthy subjects,
wherein “t” represents a degree of freedom and varies depending on the number of specimens of healthy subjects, and is selected based on a t-distribution table.

4. The method according to claim 1, wherein the determining of the amount or concentration of the solubilized podocalyxin is carried out by an immunological technique.

5. The method according to claim 1, wherein the therapeutic strategy is administering medicine to the subject suffering from diabetic nephropathy.

6. The method according to claim 5, wherein the medicine comprises a hypoglycemic agent, antihypertensive agent, and/or diuretic.

7. The method according to claim 6, wherein the hypoglycemic agent is insulin.

8. The method according to claim 6, wherein the antihypertensive agent is angiotensin-converting enzyme inhibitor, an angiotensin receptor antagonist, and/or calcium blocker.

9. The method according to claim 8, wherein the angiotensin-converting enzyme inhibitor is Perindopril, Delaprill, Trandolapril, Cilazapril, Captopril, Enalapril, Lisinopril, Benazepril, or Imidapril, the angiotensin receptor antagonist is Losartan, valsartan, Candesartan, telmisar, Olmesartan medoxomil, Irbesartan, or Azilsartan, and/or the calcium blocker is Nifedipine, Amlodipine, or diltiazem hydrochloride.

10. The method according to claim 6, wherein the diuretic is trichlormethiazide.

11. A method for treating diabetic nephropathy in a subject, comprising testing for diabetic nephropathy in the subject, comprising the steps of:
adding a treatment liquid comprising a chelating agent to a urine sample of a subject to produce a urine sample solution comprising solubilized podocalyxin;
determining an amount or concentration of solubilized podocalyxin in said urine sample solution;
calculating a urinary podocalyxin value as a diagnostic marker by using the amount or concentration of the solubilized podocalyxin and an amount or concentration of urinary creatinine;
correlating the urinary podocalyxin value with the presence of the diabetic nephropathy in said subject; and
determining a therapeutic strategy for the subject with the diabetic nephropathy and treating the subject according to the therapeutic strategy.

12. The method for treating diabetic nephropathy of claim 11, wherein the subject is a diabetic patient.

13. The method for treating diabetic nephropathy of claim 11, wherein an early stage of the diabetic nephropathy is detected, wherein said early stage is characterized by a urinary albumin (ALB) excretion rate “ALB/Cre” of less than 30 mg/g of creatinine.

14. The method according to claim 11, wherein the therapeutic strategy is administering medicine to the subject suffering from diabetic nephropathy.

15. The method according to claim 14, wherein the medicine comprises a hypoglycemic agent, antihypertensive agent, and/or diuretic.

16. The method according to claim 15, wherein the hypoglycemic agent is insulin.

17. The method according to claim 15, wherein the antihypertensive agent is angiotensin-converting enzyme inhibitor, an angiotensin receptor antagonist, and/or calcium blocker.

18. The method according to claim 17, wherein the angiotensin-converting enzyme inhibitor is Perindopril, Delapril, Trandolapril, Cilazapril, Captopril, Enalapril, Lisinopril, Benazepril, or Imidapril, the angiotensin receptor antagonist is Losartan, Valsartan, Candesartan, Telmisar, Olmesartan medoxomil, Irbesartan, or Azilsartan, and/or the calcium blocker is Nifedipine, Amlodipine, or Diltiazem hydrochloride.

19. The method according to claim 15, wherein the diuretic is Trichlormethiazide.
